(19) United States
(12) Patent Application Publication
(10) Pub. No.: US 2022/0218917 A1
(43) Pub. Date: Jul. 14, 2022

(54) METALIZED FABRIC HEATING DEVICE FOR MEDICAL SOULUTIONS

(71) Applicant: Encompass Group, LLC, McDonough, GA (US)

(72) Inventor: Mark Beran, Neenah, WI (US)

(21) Appl. No.: 17/707,773

(22) Filed: Mar. 29, 2022

Related U.S. Application Data
(63) Continuation-in-part of application No. 16/269,430, filed on Feb. 6, 2019.
Application No. 16/269,430 is a continuation-in-part of application No. 15/920,383, filed on Mar. 13, 2018.
Application No. 15/920,383 is a continuation-in-part of application No. 15/841,044, filed on Dec. 13, 2017, now Pat. No. 10,805,988.
(60) Provisional application No. 62/471,103, filed on Mar. 14, 2017.

(51) Int. Cl. A61M 5/44; A61M 16/10

(52) U.S. Cl. CPC A61M 5/445; A61M 2205/3653; A61M 16/1095

(57) ABSTRACT

There is disclosed a heating device which is adapted to be used to warm medical solutions contained within medical devices such as an intravenous bag, intravenous tube or respiratory circuit. The heating device includes a metalized fabric having a first layer of clear thermoplastic material, the second layer of vaporized aluminum material, a third layer of thermoplastic material, and a fourth layer of lofted billow spunbond thermoplastic, non-woven material. The heating device is in the form of a generally tubular sleeve forming an IV line channel therein through which the medical device.

### REFERENCE TO RELATED APPLICATION

This Application is a continuation-in-part of U.S. patent application Ser. No. 16/269,430 filed Feb. 6, 2019 and entitled “Metalized Fabric Heating Device For Medical Solutions”, which claims the benefit of U.S. Provisional Patent Application Ser. No. 62/471,103 filed Mar. 14, 2017 and entitled “Metalized Fabric Heating Blanket”, which is also a continuation-in-part of U.S. patent application Ser. No. 15/920,383 filed Mar. 13, 2018 and entitled “Metalized FabricHeating Blanket And Method Of Manufacturing Such”, which is a continuation-in-part of U.S. patent application Ser. No. 15/841,044 filed Dec. 13, 2017 and entitled “Metalized Fabric Heating Blanket”.

### TECHNICAL FIELD

This invention relates generally to heating devices, and more particularly to heating devices utilized to warm medical solutions contained within intravenous lines, intravenous bags, anesthesia and respiratory circuits, airway tubes, and the like.

### BACKGROUND OF THE INVENTION

Intravenous solutions, medications in liquid fluids, blood, plasma, and other form, collectively referenced hereinafter as solutions, are typically administered to the patient to provide select fluids or medications directly into the patient's veins. These solutions are kept in IV bags to which an IV tube or line is coupled. The opposite end of the IV tube is fitted with an IV needle or catheter which punctures the patient's skin and enters the patient's vein.

A common problem with such IV solutions and blood is that they are usually stored at room temperature or refrigerated. As such, their temperatures are far below the normal body temperature of a patient. The infusion of these cool solutions into a patient may cause discomfort, or may even lower the body temperature of the patient to the point of causing hypothermia, which has been associated with increased infection rates, cardiac instability, coagulation complications and increased overall cost to the healthcare facility.

In order to avoid this problem, medical facilities may warm the solutions prior to administering to the patient. The solution may be warmed by placing them into a warming cabinet which raises the temperature of the solution. However, the rate of the administration can allow the solutions to cool down in the IV line.

Other devices have been designed to warm the solution. One such device is a pair of warming plates between which the IV tube is positioned in a serpentine pattern to increase the contact area between the warming plates and the IV tube. A problem with this type of device is that the warming plate may cause hot spot in the IV tube causing the IV line to overheat the solutions, posing a risk to the patient.

Another device is in the form of a triple lumen tube wherein the IV line is surrounded by a circulating water jacket. Heat is exchanged between the warm water extending to the IV tube lumen in order to warm the IV solution passing through the IV tube. This type of device requires the circulation of heated water, which may cause entanglement of the lines or a spillage of the warming water. The warming line requires a circulating water bath which may present a risk of infectious material build up in the water itself if not maintained properly.

A similar problem also exists with respiratory or anesthesia circuits or airway tubes which provide air or gases to the patient. A breathing tube in a cool environment may create condensation within the tube or circuit during the breathing process, this may be referred to as “rain out”. This condensation may interfere with the proper administration of air or gases to the patient, and thus should be avoided. For ease of explanation, these gases for medical purposes are also referenced herein as “solutions” or “fluids”.

It would be beneficial to provide a device for warming IV solutions or respiratory gases to a patient which provides a safer and more consistent heat than those of the prior art. Accordingly, it is to the provision of such that the present invention is primarily directed.

### SUMMARY OF THE INVENTION

In a preferred form of the invention a heating device for warming medical solutions contained within a medical device, comprises an elongated first electrically insulative tube defining a medical solution tube channel, an elongated electrically resistive heating element tube mounted concentrically about the first electrically insulative tube, an elongated second electrically insulative tube mounted concentrically about the electrically resistive heating element tube, an elongated flexible metallic foil tube mounted concentrically about the second electrically insulative tube, and an elongated thermoplastic tube mounted concentrically about the flexible metallic foil tube.

### BRIEF DESCRIPTION OF THE DRAWINGS

FIG. 1 is a perspective view of a warming blanket embodying principles of the invention in a preferred form.

FIG. 2 is a cross-sectional view of a portion of the warming blanket of FIG. 1.

FIG. 3 is a top view of a portion of the warming blanket of FIG. 1.

FIG. 4 is a plan view of the warming blanket of FIG. 1.

FIG. 5 is a plan view of a warming blanket embodying principles of the invention in another preferred form.

FIG. 6 is a cross-sectional view of a portion of the warming blanket of FIG. 5.

FIGS. 7-12 are a series of top view of a warming blanket in another preferred embodiment, showing the manufacturing process.

FIG. 13 is a cross-sectional view of a portion of the warming blanket shown in FIGS. 7-12.

FIG. 14 is a perspective view of the heating device embodying principles of the invention in another preferred form.

FIG. 15 is a perspective view of the heating device embodying principles of the invention in another preferred form.

FIG. 16 is a perspective view of the heating device embodying principles of the invention in another preferred form.

FIG. 17 is a cross-sectional view of the heating device of FIG. 14.

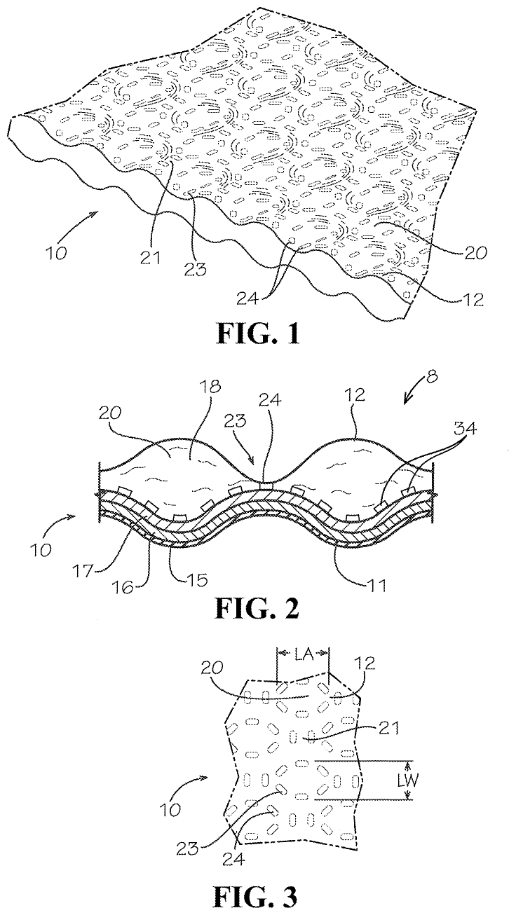

### DETAILED DESCRIPTION

With reference next to the drawings, there is shown a warming blanket 8 made in part with a metalized fabric 10 embodying principles of the invention in a preferred form. The warming blanket 8 has a lower surface 11 which is intended to face away from a person (patient) overlaid with or donning the material and an upper surface 12 which is intended to face the person (patient). The metalized fabric includes a first layer 15 of clear thermoplastic (for example a polyethylene) material, a second layer 16 of vaporized aluminum material (solid metalized layer), a third layer 17 of thermoplastic (for example a polyethylene) material, and a fourth layer 18 of lofted billow spunbond thermoplastic (for example a polypropylene) non-woven material. The exterior surface of the first layer 15 constitutes the fabric lower surface 11, while the exterior surface of the fourth layer 18 constitutes the upper surface 12.

The warming blanket 8 also includes a resistive heating portion 30 positioned between the third layer 17 and the fourth layer 18. The resistive heating portion 30 is positioned distally from the perimeter or outer edge of the warming blanket 31 and metalized fabric 10 so that a surrounding margin 32 is formed therebetween.

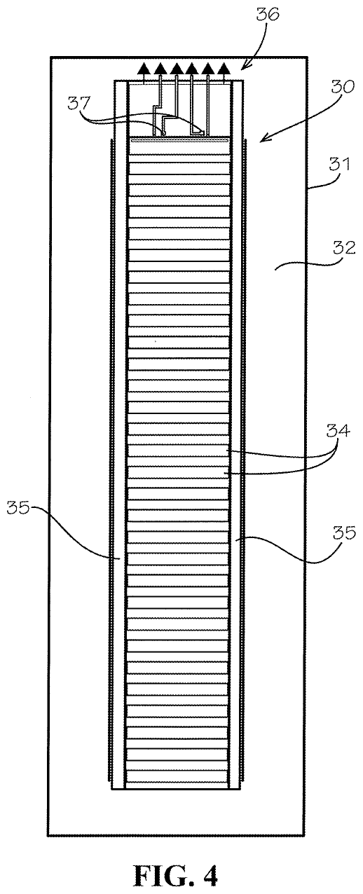

The resistive heating portion 30 has heater trace resistors or heating elements 34 arranged in a longitudinal array with each heating element 34 extending laterally, as best shown in FIG. 4. The heating elements 34 are formed by depositing a conventional electrically conductive ink upon the third layer 17 in the desired pattern. The heating elements 34 are electrically joined together through a pair of conductive tapes 35 coupled to the ends of the heating elements. The conductive tapes 35 may be made of a metal, such as copper, or in the alternative, the conductive tapes 35 may be replaced by additional conductive ink strips or any other configuration of a conductive element. The resistive heating portion 30 may also include a convention flat flex crimp pin type connectivity or coupler 36 to allow a quick connect to a controller 43, which may also include thermistors 37, or thermocouples, to regulate the current and temperature of the warming blanket 8.

The warming blanket 8 may have an input voltage of 100 to 250 VAC and a maximum blanket power of 7 W @12 VDC to 109 W@ 48 VDC.

The metalized fabric is manufactured by joining the third layer 17 of thermoplastic material having the resistive heating portion 30 thereon to the fourth layer 18 of spunbond thermoplastic non-woven material. The second layer 16 of vaporized aluminum material is then deposited or joined onto the third layer 17 via a vacuum deposit chamber. The first layer 15 is then extruded or joined onto the second layer 16. The combination of layers is then passed through cold calender rollers which seals the layers together in a pattern that forms a series, matrix or field of large pillowed areas or regions 20 surrounded at four sides by smaller pillowed regions 21. The large pillowed region 20 is generally oval in shape with a longitudinal length LA of approximately 3/16 of an inch and a lateral width LW of approximately 2/16 of an inch. The seals 23 themselves are non-continuous or fragmented, as they are formed by several unjoined segments 24 which also helps in providing a less stiff feel to the metalized fabric by breaking up the seals which tend to be stiffer than those areas of the fabric which are not sealed, i.e., the bonding of the material at the seals tends to stiffen the sealed areas and thereby tends to stiffen the overall material decreasing its drapability and loft. The metalized fabric of the present invention is fused, bonded or sealed on approximately 14% of the material, as opposed to the prior art material which included at a minimum 18% fusing, bonding or sealing.

It is believed that the position of the heating elements between the person and the metalized second layer 16 provides for a more even distribution of heat. Heat produced from the heating elements is reflected by the metalized second layer 16 back onto the person. Thus, heat initially drawn away from the person is not lost to ambient environment and is instead used to heat the person, a distinct advantage over the prior art.

It is believed that the pillowing of the metalized fabric provides for greater insulative qualities, a softer feel, better glare reduction, improved drapability, and improved loft.

Another discovered advantage has been the materials improved cross-direction tearing resistance. A test was conducted comparing the prior Thermoflect metalized material, previously described, to the metalized fabric of the present invention. The metalized fabric of the present invention was found to have a cross directional tearing factor of 435.7, while the prior Thermoflect metalized material had a tested cross directional tearing factor of 393. This test shows an improvement in tearing resistance of approximately eleven percent (11%).

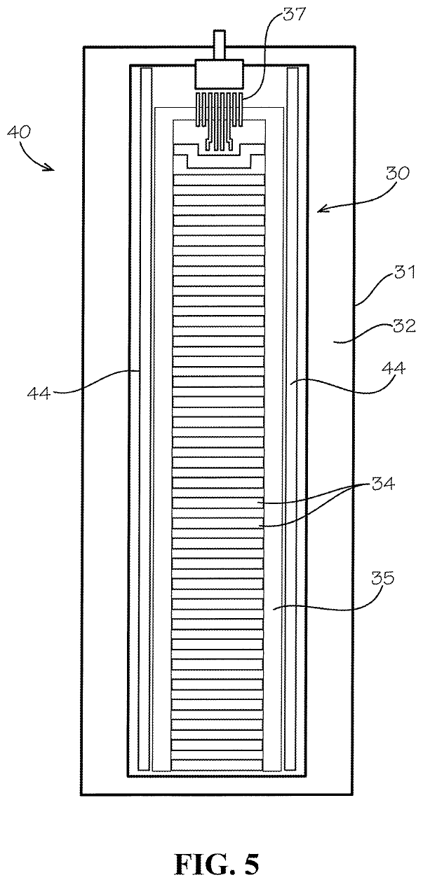

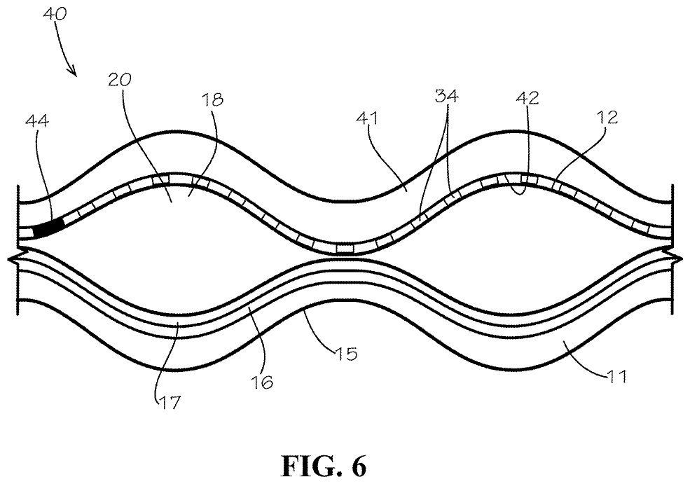

As an alternative to the first embodiment, a second embodiment of the invention in a preferred form is shown in FIGS. 5 and 6. Here, warming blanket 40 has the previously described first layer 15, second layer 16, third layer 17 and fourth layer 18 are formed as a unitary structure. A fifth layer 41 is coupled to the fourth layer 18. The fifth layer 41 may be a spunbond thermoplastic (for example a polypropylene) non-woven material. The fifth layer 41 includes the resistive heating portion 30, and especially all the previously described components including the heating elements 34 which may be in the form of electrically conductive ink, bonded or coupled to the interior surface 42 of the fifth layer 41 facing the fourth layer 18.

A pair of double-sided tape strips 44 may be applied to the fifth layer 41 so that it may be attached or coupled to a pre-existing warming blanket. Also, if need be, the fifth layer 41 with the electronic components may be easily removed or released from the warming blanket. As such, an existing warming blanket may be converted from a static or strictly body heat capturing warming blanket to a positive or active electrically resistive heat added warming blanket. The warming blanket may then be reconfigured to a static body heat capturing warming blanket by removing the fifth layer 42 and electronic components. In this manner, the electronic components may be attached and then removed from multiple warming blankets should they become soiled or otherwise unusable and may be disposed. This disposability decreases the expense involved in providing warming blankets having resistive heating capabilities.

It is believed that this embodiment provides an even higher amount of heat dispersement or distribution as a portion of the heat from the heating elements 34 initially radiating in the direction away from the patient is dispersed as it passes through the fourth layer 18, is reflected by the second layer 16, and then disperses even more as it passes again through the fourth layer 18 prior to reaching the person, i.e., the heat passes through the fourth layer 18 twice before reaching the person. This also allows the temperature of the conductive heating element 34 to be set at a lower temperature because of the additional reflected heat being directed back to the person.

It should be understood that as used herein the term “lofted” is intended to mean something that is fluffed, fluffy, expanded, expanded layers, or the like. Also, the term “billow” or “billowed” is intended to mean raised, embossed, undulating surface, having lofted areas, or the like. The use of a lofted inner material is believed to allow the heat from the heating elements 34 and that reflected back from the metalized second layer 16 to spread or diffuse the heat so as to provide a more even heating, as opposed to a concentration of the heat should a thin layer be utilized.

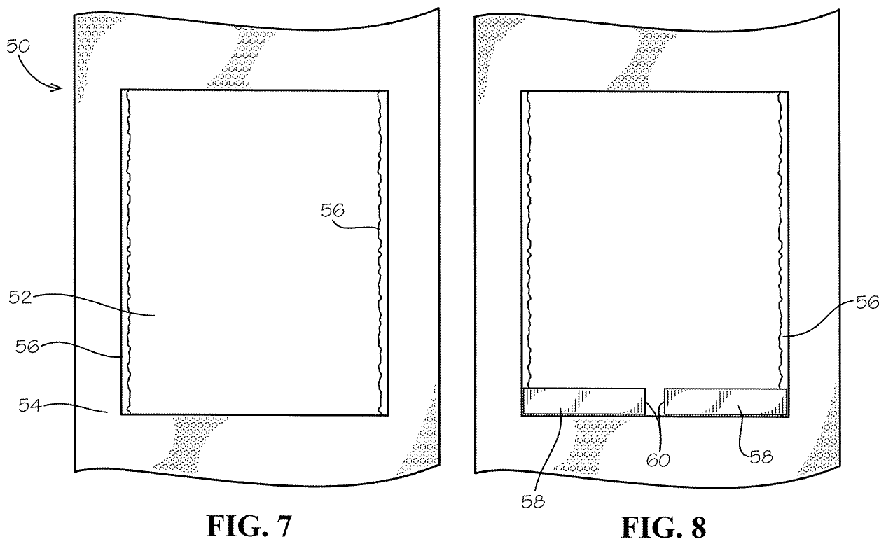

With reference next to the embodiment of FIGS. 7-13, there is shown a heating blanket 40 in another preferred form of the invention.

Here, the heating elements 34 are formed by adhering a small patch 53 of electrically insulative spunbond material to an exterior facing surface of a carbon veil material 52, wherein the carbon veil material 52 may be a sheet or mat of randomly orientated carbon fibers. The carbon veil material 52 is then adhered, through sewing, adhesive, sonic welding or the like, to a second layer of electrically insulative spunbond material 63 which will be later bonded to a previously discussed metalized fabric 54. The metalized fabric 54 is generally the same as that previously described and which includes the first layer 15 of clear thermoplastic (for example a polyethylene) material, the second layer 16 of vaporized aluminum material (metalized layer), a third layer 17 of thermoplastic (for example a polyethylene) material, and a fourth layer 18 of lofted billow spunbond thermoplastic (for example a polypropylene) non-woven material. The third layer 17 and fourth layer 18 may also be electrically insulative.

Next, a conductive strip in the form of a conductive ink layer 56, which may be made of nickel or silver ink, is deposited, sprayed upon, or printed onto opposite side edges of the carbon veil material 52 as thin strips or side rails 56, also shown in FIG. 7. The conductive ink side rails 56 acts to locally connect the random conductive fibers at different depth of the carbon veil material 52.

With reference next to FIG. 8, lower conductive strips 58 are then sewed on, or alternatively attached by electrically conductive adhesive or other bonding method, onto a bottom edge of the carbon veil material 52. Each lower conductive strip 58 is electrically coupled to a side rail 56. The lower conductive strips 58 may be made of an aluminum foil or other electrically conductive material. The lower conductive strips 58 are electrically insulated from the carbon veil material 52. The lower conductive strips 58 have connecting ends 60 which are spaced from each other so as to accept a connection circuit board described in more detail hereinafter.

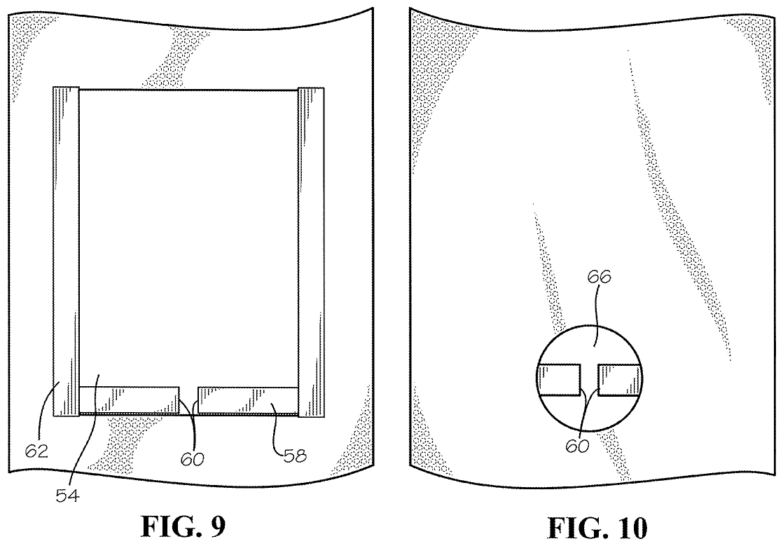

With reference next to FIG. 9, side conductive strips 62 are then sewed onto the conductive ink side rails 56 in electrical contact with the conductive ink side rails 56. The nickel boundary of the conductive ink side rails 56 prevent resistance drift from occurring. The side conductive strips 62 are also sewn so as to be in electrical contact with the lower conductive strips 58.

The second layer of spunbond material 63 is then laminated or otherwise bonded (adhesive, sonic welding, or the like) about the periphery of the fourth layer (spunbond material) 18 and/or carbon veil material 52, thereby sandwiching the carbon veil material 52 between two layers of spunbond material. The second layer of spunbond material 63 protects the carbon veil material 52 while providing a soft exterior layer for patient comfort and safety. The combination of the second layer of spunbond material 63 with the first layer of spunbond material (metalized fabric) essentially creates an envelope surrounding or encasing the carbon veil.

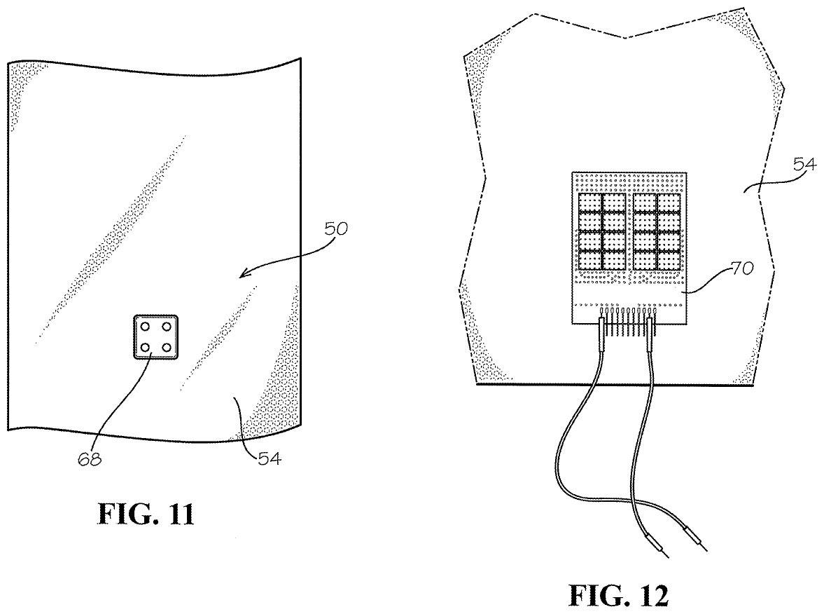

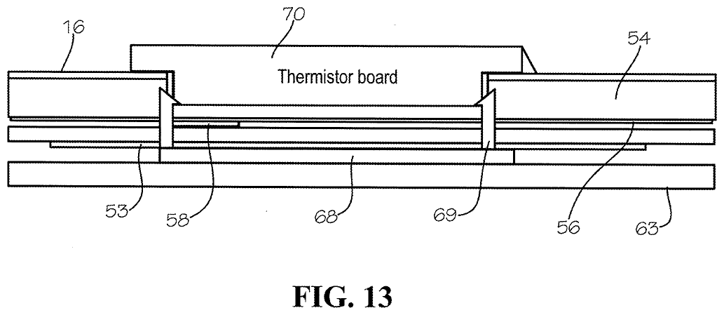

With reference next to FIG. 10, a hole or opening 66 is cut into the metalized fabric 54 so as to expose the connecting ends 60 of the lower conductive strips 58. A backing plate 68 is then attached to the backside of the second layer of spunbond material 63 at the position of the opening 66, as shown in FIG. 11, or to a patch of spunbond material which is then adhered to the patient side of the blanket. The backing plate 68 may be passed through a slot or cut 67 in the second layer of spunbond material 63 so as to be placed flush against the patch 53, as shown in FIG. 13. The use of the backing plate 68 provides local support of the connection points of the warming blanket as well as providing pressure between the contact surfaces of the thermistor board and the lower conductive strips 58 (cross rails). The backing plate 68 includes a set of mounting prongs 69 which extend through or are punched through the patent 53 and carbon veil material 52 so that they may engage, fit upon a snap-on circuit board 70 containing thermistors (thermistor plate 71), or thermocouples. The circuit board 70 is then mounted to the exterior surface of the metalized fabric 54 and connected to the connecting ends 60 of the lower conductive strips 58, as shown in FIGS. 12 and 13. The circuit board 70 includes a large array of vias to assist heat transfer to the where the thermistors are located. The use of a large circuit board for connection purposes provides a more accurate average temperature of the heating fabric (carbon veil material), i.e., the temperature is sensed over a larger area for averaging purposes to minimize the possibility of errors. The vias transfer heat to the top side of the circuit board so that the thermistors can be captured within the connector housing. This also shields the thermistors for the safety of the operator.

In use, electric current is controlled through the circuit board 70 and passed to the connecting ends 60 of the lower conductive strips 58. The current then travels to the side conductive strips 62 and conductive ink side rails 56 where it is then passed to the carbon veil material 52 wherein resistive heat is created. The metalized fabric reflects the heat to produce an even distribution and more efficient use of the heat. The lofted material layers diffuse the heat to avoid a concentration of heat or hot spot.

The circuit board 70 uses multiple thermistors to minimize variance. The placement of the thermistors on the circuit board 70 enables them to be on a re-useable portion of the warming blanket 50 rather than the disposable “blanket” or material covering portion. This placement reduces the replacement costs of the warming blanket.

It is believed that the sewing of the conductive foil of the lower conductive strips 58 and side conductive strips 62 to the second layer of spunbond material 63 and carbon veil material 52 provides a better electrical connection. It is also believed that the sewing maintains a better drape ability of the warming blanket. The improved drape ability is important for patient comfort, effective warming, and reduced cost of manufacture.

The sewing process of the lower conductive strips 58 and the side conductive strips 62 preferably is accomplished with the use of non-conductive cotton-poly blend threads.

It should be understood that the description is for one method of constructing the warming blanket. The exact sequence of the steps involved in the construction may differ while still embodying the invention.

It should be understood that sewing, adhesive bonding, sonic welding, heat welding, or any other conventional method of bonding or coupling, as used herein, are equivalent.

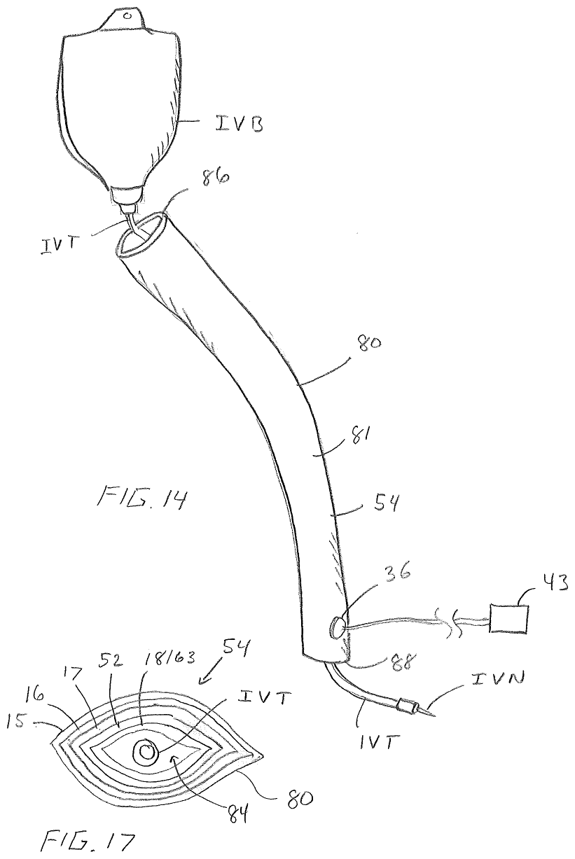

With reference next to FIG. 14, there is shown a warming device or heating device 80 which is adapted to be used to warm medical solutions contained within an intravenous tube or IV line IVL. As the warming device or heating device 80 is utilized in conjunction with other medical devices (IV line) containing a solution, it is referenced hereinafter as a heating device 80 for medical solutions.

The heating device 80 uses the same construction method described in the previous embodiments. As such, the heating device includes the previously described metalized fabric 54 having an elongated, tubular form of the metalized or metallic layer 16 and spunbond material 18 or 63. Specifically, the metalized fabric 54 includes an elongated tube of the first layer 15 of clear thermoplastic (for example a polyethylene) material, an elongated tube of the second layer of vaporized aluminum material (metalized layer), an elongated tube of the third layer 17 of thermoplastic (for example a polyethylene) material, and an elongated tube of the fourth layer 18 or 52 of lofted billow spunbond thermoplastic (for example a polypropylene) non-woven material. An elongated tube of the electrically resistive heating element in the form of the previously described carbon veil 52 is positioned concentrically between the third layer 17 and the fourth layer 18/63. The third layer may also be electrically insulative. Here, the heating device 80 is in the form of a generally tubular sleeve wherein each previously described layer is mounted concentrically to the inboard surface of the previous layer so as to form a sleeve with concentric layers, the term “inboard” meaning the inside or interior surface of the tubular form, rather than the outboard, exterior, or outside surface of the tubular form.

It should be understood that while this is a preferred orientation, the warming device layers may be oriented in reverse order with the first layer 15 as the innermost layer. However, this is not preferred as it is believed that by orienting the metallic layer 16 as the outermost layer with respect to the carbon veil 52 the heat produced by the carbonveil is reflected back towards the IV line IVL to provide a consistent and efficient heating of the IV solution.

The heating device metalized fabric 54 is formed as an elongated flexible material tube or sleeve 82 having an interior IV line or central channel 84 therein extending from a first end 86 to a second end 88. The first end 86 is positioned closely adjacent the connection between an IV solution bag IVB and the IV line IVL. The second end 88 is positioned adjacent the terminal end or coupler end to the IV needle IVN of the IV line IVL.

The flexible material tube 82 has the vaporized aluminum material (solid metalized layer, or second layer 16) of the metalized fabric 54 facing outwardly and the spunbond material 63 facing inwardly towards the IV line IVL. The metalized fabric 54 is coupled to an electrical controller 43 through an electrical coupler 36 as previously described. The controller 43 may adjust the current passed to the carbon veil 52 to regulate or control the temperature produced by the heating device 80.

In use, the IV line IVL is passed through the heating device channel 84 so that the heating device 80 covers at least a majority of the IV line IVL.

With the heating device 80 activated, the heat produced by the current controlled by controller 43 and passing through the carbon veil 52 is directed towards the heating device channel 84 and the IV line IVL therein. This heat warms the solution contained within the IV line IVL prior to the solution entering the patient through the IV needle or catheter IVN coupled to the IV line IVL. The heat produced by the carbon veil 52 is also reflected by the vaporized aluminum material (second layer 16) back towards the carbon veil 52 so that more of the heat is directed to the IV line IVL positioned within the central channel 84. This reflected heat also produces a greater efficiency and a more even distribution of the heat, which avoids the creation of hot spots or the like.

The quantity of heat produced by the heating device 80, or its temperature, is regulated through the previously described controller 43 and coupler 36. The quantity of heat may be tied to or regulated according to the flow rate of the solution through the IV line IVL, i.e., the faster the fluid flows through the IV line the higher the temperature of the heating device 80 should be set to compensate for the shorter time the fluid passes through the heating device 80. This correlation between the flow rate of the solution and the flow of current from the controller 43 may be an automated program wherein the controller is in communication with the flow rate mechanism or controller of the IV solution, or may be manually controlled through a manual activation or desired temperature adjustment of the controller 43.

It should be understood that the tubular sleeve of the heating device may be formed with fasteners on opposite ends of a sheet type form of the device which may be fastened together to form a tube, or otherwise fastened together or positioned to form a tube. This will enable the device to be wrapped about or mounted to the IV line IVL and then fasten the ends together or overlapping fashion even after the needle IVN is inserted into a patient, i.e., it does not require that the IV tube be threaded through a tubular structure or central channel 84 prior to the IV needle IVN being inserted into the patient.

With reference next to FIG. 15, it should be understood that a heating device 100 of the present invention in another form may be configured, as previously described, to surround, or at least partially surround, the IV bag IVB, alone or in addition to surrounding the IV line IVL.

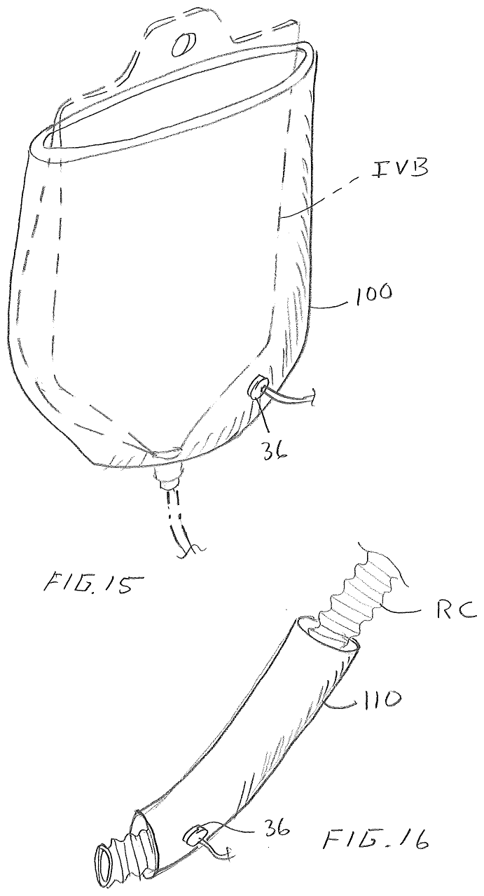

With reference next to FIG. 16, a heating device 110 in another form may be configured to surround a respiratory tube or circuit RC, rather than an IV line, as shown in FIG. 16. By surrounding the respiratory tube, the gases within the respiratory tube are warmed to prevent condensation within the respiratory tube. As such, it should be understood that the present heating device may be utilized with many different types of medical devices, including but not limited to medical tubes, lines, bags, or other devices to maintain them and the solutions (fluids, gases, liquids, etc.) flowing through them to the patient in a warm state.

It should be understood that in the embodiments of FIGS. 14-17, the spunbond material 63 may be eliminated as the heating device 80 does not come into contact with a patient. However, the spunbond material 63 is still preferred as it helps to diffuse the heat for a more even heating of the medical device within the present heating device.

Thus, a heating device for warming medical solutions contained within a medical device, comprises an elongated first electrically insulative tube defining a medical solution tube channel, an elongated electrically resistive heating element tube mounted concentrically about or surrounding the first electrically insulative tube, an elongated second electrically insulative tube mounted concentrically about or surrounding the electrically resistive heating element tube, an elongated flexible metallic foil tube mounted concentrically about or surrounding the second electrically insulative tube, and an elongated thermoplastic tube mounted concentrically about or surrounding the flexible metallic foil tube.

It thus is seen that a metalized fabric heating device for medical solutions is now provided which overcomes problems associated with heating device of the prior art. It should of course be understood that many modifications may be made to the specific preferred embodiment described herein, in addition to those specifically recited herein, without departure from the spirit and scope of the invention as set forth in the following claims.

1. A heating device for warming medical solutions contained within a medical device, comprising:
an elongated first electrically insulative tube defining a medical solution tube channel;
an elongated electrically resistive heating element tube mounted concentrically about said first electrically insulative tube;
an elongated second electrically insulative tube mounted concentrically about said electrically resistive heating element tube;
an elongated flexible metallic foil tube mounted concentrically about said second electrically insulative tube, and
an elongated thermoplastic tube mounted concentrically about said flexible metallic foil tube.

2. The heating device of claim 1 wherein said electrically resistive heating element tube is a carbon veil tube.

3. The heating device of claim 1 further comprising an electrical control circuit electrically coupled to said electrically resistive heating element tube.

4. The heating device of claim 1 further comprising a controller to control the flow of electric current to said electrically resistive heating element tube.

5. The heating device of claim 4 wherein said controller receives a signal indicating the flow of a medical solution passing through said medical solution tube channel and said controller controls the electric current to said electrically resistive heating element tube in accordance with the received flow of a medical solution passing through said medical solution tube channel.

6. A heating device for warming medical solutions contained within a medical device, comprising:
an elongated first electrically insulative tube defining a medical solution device channel;
an elongated electrically resistive heating element tube surrounding said first electrically insulative tube;
an elongated second electrically insulative tube surrounding said electrically resistive heating element tube;
an elongated flexible metallic foil tube surrounding said second electrically insulative tube, and
an elongated thermoplastic tube surrounding said flexible metallic foil tube.

7. The heating device of claim 6 wherein said electrically resistive heating element tube is a carbon veil tube.

8. The heating device of claim 6 further comprising an electrical control circuit electrically coupled to said electrically resistive heating element tube.

9. The heating device of claim 6 further comprising a controller to control the flow of electric current to said electrically resistive heating element tube.

10. The heating device of claim 9 wherein said controller receives a signal indicating the flow of a medical solution passing through said medical solution device channel and said controller controls the electric current to said electrically resistive heating element tube in accordance with the received flow of a medical solution passing through said medical solution device channel.

11. A heating device for warming medical solutions contained within a medical device, comprising:
a first electrically insulative tube defining a central channel;
an elongated electrically resistive heating element tube mounted about said first electrically insulative tube;
an elongated second electrically insulative tube mounted about said electrically resistive heating element tube;
an elongated flexible metallic foil tube mounted about said second electrically insulative tube, said metallic foil tube being heat reflective to reflect heat produced by said electrically resistive heating element back towards said electrically resistive heating element and towards said central channel of said first electrically insulative tube, and
an elongated thermoplastic tube mounted about said flexible metallic foil tube.

12. The heating device of claim 11 wherein said electrically resistive heating element tube is a carbon veil tube.

13. The heating device of claim 11 further comprising an electrical control circuit electrically coupled to said electrically resistive heating element tube.

14. The heating device of claim 11 further comprising a controller to control the flow of electric current to said electrically resistive heating element tube.

15. The heating device of claim 14 wherein said controller receives a signal indicating the flow of a medical solution passing through said central channel and said controller controls the electric current to said electrically resistive heating element tube in accordance with the received flow of a medical solution passing through said central channel.
